(19) United States
(12) Patent Application Publication
(10) Pub. No.: US 2002/0120147 A1
(43) Pub. Date: Aug. 29, 2002

(54) Micronized torsemide

(76) Inventors: Marco Kordova, Kfar Saba (IL); Anchel Schwartz, Rechovot (IL); Ben-Zion Dolitzky, Petach Tikva (IL); Judith Aronhime, Rechovot (IL); David Leonov, Rechovot (IL); Shlomo Zavurov, Lod (IL)

Correspondence Address: KENYON & KENYON, ONE BROADWAY, NEW YORK, NY, 10004, US

(21) Appl. No.: 09/929,410

(22) Filed: Aug. 14, 2001

Related U.S. Application Data
(60) Provisional application No. 60/225,365, filed on Aug. 14, 2000.

(51) Int. Cl. C07D 211/84; C07D 213/62

(52) U.S. Cl. 546/291

(57) ABSTRACT

The present invention relates to torsemide having relatively small particles, and corresponding relatively large surface area. In one embodiment, the invention relates to torsemide and formulations containing torsemide having a mean particle diameter of less than 200 micrometers.

### CROSS-REFERENCE TO RELATED APPLICATION

The present application claims benefit of U.S. provisional application Ser. No. 60/225,365, filed Aug. 14, 2000, which is incorporated by reference.

### FIELD OF THE INVENTION

This invention relates to micronized torsemide and to the preparation thereof.

### BACKGROUND OF THE INVENTION

1-Isopropyl-3-[(4-m-toluidino-3-pyridyl) sulfonyl] urea, which has the chemical structure

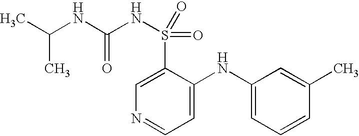

is approved, under the trademark DEMADEX®, by the U.S. Food and Drug Administration for the treatment of hypertension and edema associated with congestive heart failure, renal disease, or hepatic disease. The USAN approved generic name for this compound is torsemide, although this compound is also referred to as “torasemide” in the art. Torsemide is a loop diuretic that has been found to be particularly effective for the treatment of edema associated with chronic renal failure.

U.S. Pat. No. Re. 30,633, the contents of which are incorporated by reference, describes a synthesis of torsemide. The references, Acta Cryst. 1978, pp. 2659-2662; Acta Cryst., 1978, pp. 1304-1310;and Pharmaceutical Dosage Forms: Tablets, Vol. 2, 2^{nd} Ed., Lieberman et al. Ed., Marcel Dekker, Inc, N.Y., (1990) p.107-200; and U.S. Pat. Nos. 4,822,807, and Re. 34,672 are all incorporated herein by reference.

Torsemide is essentially insoluble in water. The Particle Size Distribution (PSD) of torsemide crystals may be used to determine the available surface area for the drug dissolution. Often, it is observed that the available surface area for drug dissolution correlates to both (a) the rate of dissolution and solubility where a greater surface area enhances the solubility of a drug; and (b) enhances the rate of dissolution of a drug. Further, the velocity of dissolution of a drug often effects the drug's bioavailability. Thus, the PSD of torsemide, and in particular, the mean particle diameter, are important parameters to characterize and predict the bioavailability of the drug. It is desirable to have torsemide with a particle size in which the mean particle size enhances the reproducibility of; (a) the rate of dissolution and (b) the reproducibility of the dissolution. It is desirable to have torsemide in which the mean particle size imparts an improved and stable dissolution profile.

### SUMMARY OF THE INVENTION

An object of the present invention is to provide torsemide formulations containing torsemide having relatively small particles, and corresponding relatively large surface area.

It is also an object of the present invention is to provide torsemide with a particle size in which the mean particle size enhances the reproducibility of; (a) the rate of dissolution and (b) the reproducibility of the dissolution.

It is also an object of the present invention to provide torsemide in which the mean particle size imparts an improved and stable dissolution profile.

The present invention provides torsemide and formulations containing torsemide having a mean particle diameter of less than 200 micrometer.

The present invention also provides torsemide and formulations containing torsemide having a mean particle diameter of less than 20 micrometers.

The present invention also provides processes for preparing micronized torsemide.

The present invention also provides processes for preparing micronized torsemide, where in the torsemide to be micronized is pure torsemide.

The present invention also provides processes for preparing micronized torsemide, where in the torsemide to be micronized is dry torsemide.

The present invention also provides pharmaceutical compositions comprising micronized torsemide.

### DETAILED DESCRIPTION OF THE INVENTION

The present invention provides torsemide formulations containing torsemide having relatively small particles, and corresponding relatively large surface area.

The present invention also provides torsemide and formulations containing torsemide having a mean particle diameter of less than 200 micrometer, preferably the mean particle diameter is less than 100 microns, more preferably the mean particle diameter is less than 20 microns, most preferably the mean particle size is about 10 microns.

The present invention also provides torsemide having a mean particle diameter of between about 200microns and about 10 microns. In another embodiment of the present invention, torsemide has a mean length of about 4.2 microns, more preferably a mean length of 4.0 microns. The term “micronized” is used herein as referring to particles having a mean particle diameter of less than about 200 microns. Micronized particles of torsemide, may be obtained by methods disclosed in U.S. Pat. No. 5,834,472, the contents of which are incorporated herein by reference.

The present invention also provides processes for preparing micronized torsemide. By the methods of the present invention, torsemide, which is prepared by methods known in the art, is separated by sieves to produce torsemide wherein 50% has a mean particle diameter of below about 250 microns and about 80% has is below about 500 microns. The sieved torsemide is then micronized by methods known in the art, e.g., in a micronizer, to yield torsemide wherein 100% of the torsemide has a mean particle size of less than about 45 microns, preferably 99% of the torsemide has a mean particle size of less than about 45 microns, more preferably, 93% of the torsemide has a mean particle size of less than about 7.5 microns, more preferably the torsemide isolated has a mean particle diameter of less than 10 micron.

Micronized particles of torsemide can be obtained by the use of conventional micronizing techniques after sieving to provide torsemide wherein about 50% has a particle size less than 250 microns and about 80% has a particle sized below about 500 microns. By the methods of the present invention, the torsemide where about 50% has a particle size less than 250 microns and about 80% has a particle sized below about 500 microns, is micronized to the desired particle size range by methods known in the art, for example, using a ball mill, ultrasonic means, fluid energy attrition mills, or using a jet mill, or other suitable means as disclosed in Pharmaceutical Dosage Forms: Tablets, Vol. 2, ₂^{nd} Ed., Lieberman et al. Ed., Marcel Dekker, Inc, N.Y., (1990) p. 107-200, the contents of which are incorporated herein by reference.

The present invention also provides processes for preparing micronized torsemide, wherein the micronized torsemide is made from dry torsemide.

In accordance with the present invention, the present micronized torsemide may be prepared as pharmaceutical compositions that are particularly useful for the treatment of hypertension and edema associated with congestive heart failure, renal disease, or hepatic disease. Such compositions comprise micronized torsemide with pharmaceutically acceptable carriers and/or excipients known to one of skill in the art.

Preferably, these compositions are prepared as medicaments to be administered orally, or intravenously. Suitable forms for oral administration include tablets, compressed or coated pills, dragees, sachets, hard or gelatin capsules, sub-lingual tablets, syrups and suspensions. While one of ordinary skill in the art will understand that dosages will vary according to the indication, age of the patient, etc., generally micronized torsemide of the present invention will be administered at a daily dosage of about 2 to about 200 mg per day, and preferably about 5 to about 100 mg per day.

### EXAMPLES

The present invention will now be further explained in the following example. However, the present invention should not be construed as limited thereby. One of ordinary skill in the art will understand how to vary the exemplified preparations to obtain the desired results.

### Example 1

Pure dry torsemide was micronized in a micronizer. The result was micronized torsemide of a particle size of less than 5 micrometer.

It should be understood that some modification, alteration and substitution is anticipated and expected from those skilled in the art without departing from the teachings of the invention. Accordingly, it is appropriate that the following claims be construed broadly and in a manner consistent with the scope and spirit of the invention.

What is claimed is:

1. Micronized torsemide.
